(19) United States
(12) Patent Application Publication
(10) Pub. No.: US 2013/0029286 A1
(43) Pub. Date: Jan. 31, 2013

(54) DEVICES AND METHODS FOR CONFORMING PHOTODYNAMIC THERAPY TO SPECIFIC ANATOMIC LOCATIONS

(71) Applicants: Thomas H. FOSTER, Rochester, NY (US); Jannick P. Rolland, Pittsford, NY (US); Florian R. Fournier, Rochester, NY (US); Cristina Canavesi, Rochester, NY (US)

(72) Inventors: Thomas H. FOSTER, Rochester, NY (US); Jannick P. Rolland, Pittsford, NY (US); Florian R. Fournier, Rochester, NY (US); Cristina Canavesi, Rochester, NY (US)

(21) Appl. No.: 13/567,605

(22) Filed: Aug. 6, 2012

Related U.S. Application Data
(63) Continuation-in-part of application No. PCT/US11/23717, filed on Feb. 4, 2011.
(60) Provisional application No. 61/301,389, filed on Feb. 4, 2010.

(51) Int. Cl. A61N 5/06; A61C 9/00

(52) U.S. Cl. 433/29; 433/217.1

(57) ABSTRACT

A device for conforming photodynamic therapy to a specific anatomic location (e.g., in the oral cavity) conforms the radiation to the target tissue surface and avoids delivering light to the rest of the oral cavity. Embodiments can include a body of oral impression material molded to conform to the anatomic surface, a light pipe, a freeform reflector formed on a surface of the optical body, or a light guide having a textured surface to direct light to an opposing output window. The light guide can be made of flexible plastic to conform the output window to the lesion.

### REFERENCE TO RELATED APPLICATIONS

The present application is a continuation-in-part of PCT/US2011/023717, filed Feb. 4, 2011, published on Aug. 11, 2011, as WO 2011/097458, which claims the benefit of U.S. Provisional Patent Application No. 61/301,389, filed Feb. 4, 2010. The disclosures of the above applications are hereby incorporated by reference in its entirety into the present disclosure.

### STATEMENT OF GOVERNMENT INTEREST

This invention was made with government support under Grant Nos. CA68409, CA122093 and CA55791 awarded by National Institutes of Health. The government has certain rights in the invention.

### FIELD OF THE INVENTION

The present invention is directed to devices for photodynamic therapy (PDT) and more particularly to such devices for irradiating complex surfaces of the oral cavity and possibly other anatomic sites. The present invention is further directed to the corresponding methods for making and using such devices.

### DESCRIPTION OF RELATED ART

Currently, PDT of cancer and precancer and other lesions within the oral cavity is performed using lens-terminated optical fibers that deliver a cone of light in the forward direction. Such fibers are often handheld and pointed by the physician to the target area. Surfaces near the back of the tongue are very difficult to irradiate in that way. Irregular surfaces along gum lines and under the tongue typically receive non-uniform irradiation that is extremely difficult or impossible to characterize and leads to imprecise and irreproducible dosimetry. Further, light reflected from the irradiated surface exposes normal tissue in the mouth to light, which leads to normal tissue damage unless those areas are carefully shielded prior to irradiation. That is very time consuming and adds significantly to the cost and complexity of the procedure.

### SUMMARY OF THE INVENTION

A need thus exists in the art to solve both of the above problems.

It is therefore an object of the invention to provide such a solution.

It is another object of the invention to provide optimal administration of photodynamic therapy irradiation to complex surfaces of the oral cavity.

It is still another object of the invention to provide an approach that may be suitable to other anatomic sites where conforming the treatment field and shielding nearby normal tissues are important.

To achieve the above and other objects, the present invention is directed to various embodiments of PDT in which the irradiation is conformed to the target tissue surface. A device for conforming photodynamic therapy to a specific anatomic location (e.g., in the oral cavity) conforms the radiation to the target tissue surface and avoids delivering light to the rest of the oral cavity. A first embodiment has a body of oral impression material molded to conform to the anatomic surface, a light source at least partially embedded in body of oral impression material and a reflective surface formed on the body of oral impression material. A second embodiment has a light pipe comprising a straight section, a reflective coating on the straight section and an output window on the straight section, the light pipe having an input end, and a light source connected to the input end. A third embodiment has a light source, an optical body having an output window and a hole for insertion of the source, and a freeform reflector formed on a surface of the optical body. In fourth and fifth preferred embodiments, one surface of the light guide is textured to direct light to an output window on the opposing side. In those embodiments, the light guide can be flexible to conform the output window to a lesion to be treated.

By conforming the irradiation to the target tissue surface, the PDT light dose becomes more uniform and more reproducible and is much easier to characterize. The device makes direct contact with the target tissue surface, and the sides and back of the device are coated with reflecting material. Therefore, there is minimal or no light delivered to the healthy surrounding tissue. That will minimize or eliminate the need for shielding normal tissue during treatment.

The invention enables more uniform and reproducible irradiation of specific tissue surfaces and eliminates or greatly reduces the need for shielding of normal tissue in the oral cavity and other anatomic sites, including those where irregular or difficult to reach surfaces create significant challenges to clinicians.

Any of the preferred embodiments, or any other embodiment, can be implemented as a “use once” disposable device. Also, the present invention can be used in dermal applications to treat stubborn spots or in cases in which only a determined region needs to be treated.

### BRIEF DESCRIPTION OF THE DRAWINGS

Preferred embodiments of the invention will now be set forth in detail with reference to the drawings, in which:

FIG. 1 shows a device according to a first preferred embodiment;

FIG. 2 shows a device according to a second preferred embodiment;

FIG. 3 shows a device according to a third preferred embodiment;

FIG. 4 shows a device according to a fourth preferred embodiment; and

FIG. 5 shows a device according to a fifth preferred embodiment.

### DETAILED DESCRIPTION OF THE PREFERRED EMBODIMENTS

Preferred embodiments of the present invention will now be set forth in detail with reference to the drawings, in which like reference numerals refer to like elements or steps throughout. Five preferred embodiments will be disclosed, although those skilled in the art who have reviewed the present disclosure will readily appreciate that other embodiments can be realized.

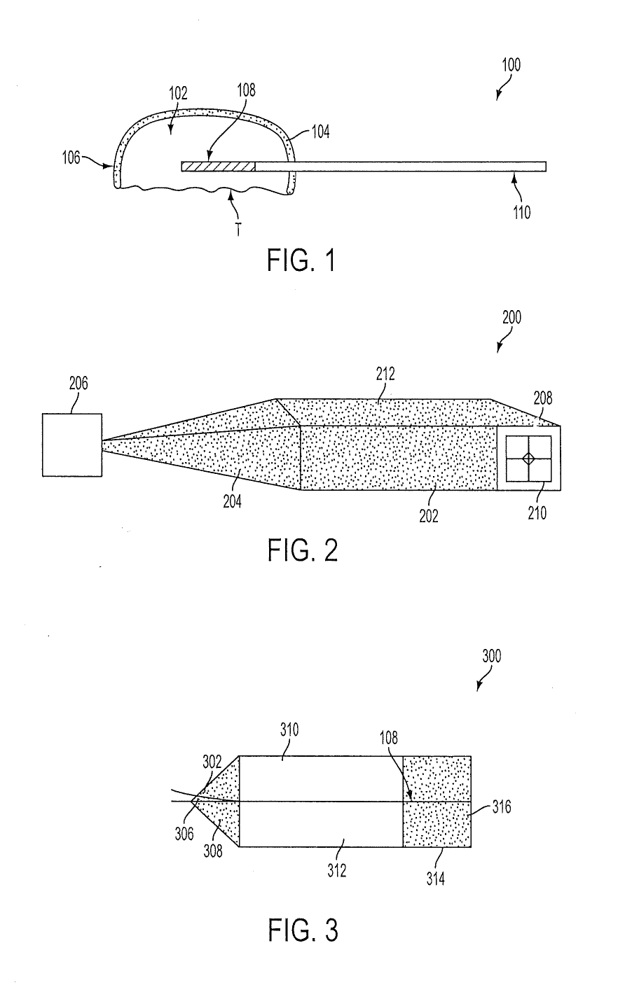

The first preferred embodiment, shown in FIG. 1 as 100, exploits a combination of oral impression materials and cylindrical diffusing tip optical fibers. Oral impression materials are available commercially and are used routinely to create molds of surfaces in the oral cavity. For example, those materials may be used to create a mold for an oral prosthesis.

In the first preferred embodiment, the material 102 is introduced into the oral cavity at the site to receive photodynamic therapy. The material hardens and assumes the shape of the tissue surface T intended to receive the PDT. It is then removed, and surfaces 104 of the impression material 102 not contacting the tissue T to be treated are coated with highly reflecting material 106 to eliminate irradiation of normal tissue and direct the light back to the treated tissue surface T.

To enable light delivery, a cylindrical diffusing tip optical fiber 108, a linear array of light emitting diodes, or another suitable source is introduced into the oral impression material 102 at the time it is introduced into the oral cavity—that is, prior to hardening. Thus, the light delivery source 108 is embedded into the form-fitting oral impression material, and a connection 110 to a laser or an LED power supply (not shown) is also provided. The light delivery source 108 embedded into the oral impression material 102 is envisioned as a “use once” disposable. Other geometries are straightforward extensions of that idea.

The second preferred embodiment uses lightpipes to achieve both high illumination uniformity and efficiency. Lightpipes are routinely used in non-imaging optics, but they require significant expertise and design effort to achieve the required illumination quality given specific application constraints.

FIG. 2 shows a device 200 according to the second preferred embodiment. The device 200 comprises a straight lightpipe section 202 and an optional tapered lightpipe section 204. This configuration is illustrative rather than limiting; other configurations may include a straight section, a tapered section, a freeform section, or any combination of these. A light source 206 such as a light-emitting diode or an optical fiber illuminator is connected to the input end of the device. The shape of the opposite end 208 of the lightpipe is tailored to extract light outside the device and deliver uniform illumination into the oral cavity. Another possibility is that light exits directly from that side without being reflected to the side window. In the example shown, the lightpipe end 208 is cut at an angle so that light exits the device through a side window 210. The lightpipe length and shape are optimized to homogenize light and provide high illumination uniformity at the device output. The tapered lightpipe section 204, which is optional, provides improved compactness.

The device 200 can be either hollow or solid and, as noted above, can be composed of a straight section, a tapered section, a freeform section, or any combination of these. Hollow lightpipes use reflective material 212 on the lightpipe walls to guide light within the lightpipe. Solid lightpipes can be made of glass or plastic and use either reflective coatings or total internal reflection to contain light within the lightpipe. If a reflective coating is used, a window such as the window 210 depicted in FIG. 2 must be left uncoated so that light can exit the device.

The device 200 simply needs to be connected to a fixed light source 206 and is envisioned as a “use once” disposable. Alternatively, a removable and disposable jacket covering the device can be used if multiple uses are desired.

The third preferred embodiment, shown in FIG. 3 as 300, uses a tailored freeform reflector shape 302 to provide uniform illumination in the oral cavity. Light delivery is achieved with an array of light-emitting diodes or a cylindrical fiber diffuser 108 similar to the one used in the first preferred embodiment. The source 108 is inserted through a hole 306 in the sidewalls 308 of the device 300 to allow precise positioning of the source 108 relative to the reflector 302. The device 300 is hollow, but a material 310 with scattering properties may be used to fill the device 300 and improve illumination uniformity. All surfaces 314 are reflective except a transparent window 312 that allows light to be extracted outside the device 300. In the example shown, the window 312 is placed at the extremity of the front end 316 of the device 300, but its position and size may vary.

The back end of the device 300 is made of the freeform reflector shape 302 optimized to obtain high light extraction efficiency and high spatial illumination uniformity. The shape is optimized from a first-order shape sometimes used for solar concentrators with cylindrical absorbers. That first-order shape is derived using the general edge-ray principle of non-imaging optics. Provided an optimal placement of the source with respect to the reflector, cylindrical reflectors may also be optimized.

The device 300 is envisioned as a “use once” disposable. Alternatively, a removable and disposable jacket covering the device can be used if multiple uses are desired.

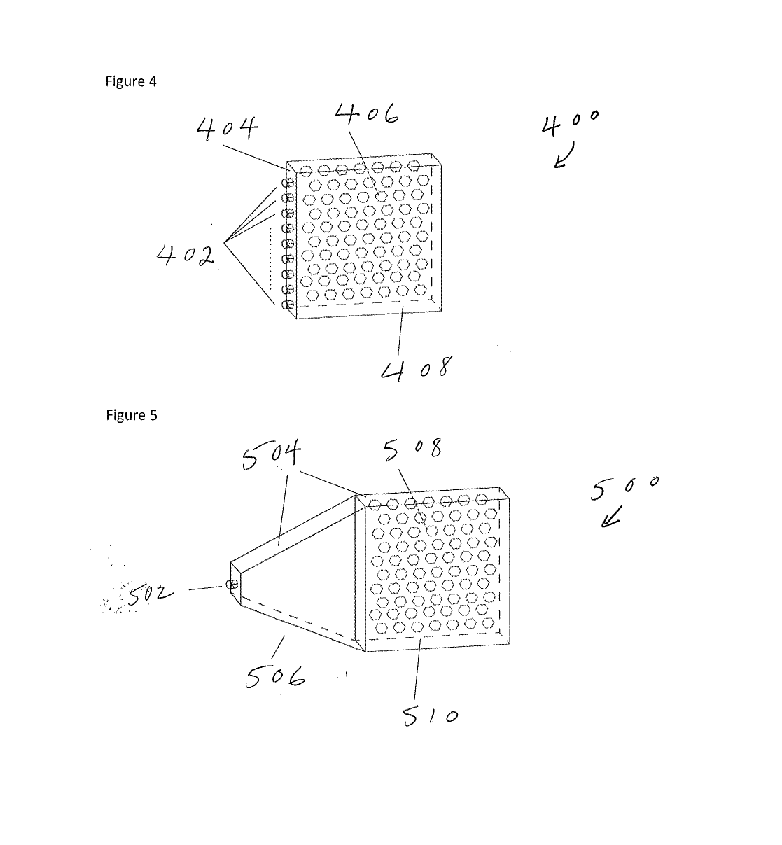

The fourth preferred embodiment, shown in FIG. 4 as 400, is based on an approach similar to those used in the design of backlight displays to provide a targeted illumination with known dosimetry. Light from an array 402 of optical fibers connected to a light source (such as a laser, not shown) or an array of light emitting diodes is coupled to a light guide 404 made of plastic or another suitable material. One side 406 of the light guide 400 is textured in an appropriate way to cause light extraction from the light guide 400 towards the side 408 opposite to the textured side, referred to as the output window.

A fifth preferred embodiment, similar to the fourth preferred embodiment, is shown in FIG. 5 as 500. An optical fiber 502 connected to a light source (such as a laser, not shown) or a light emitting diode is coupled to a light guide 504 made of plastic or another suitable material. An initial portion 506 of the light guide 504 may optionally be tapered to increase the size of the light guide. One side 508 of the light guide is textured (partially or entirely) in an appropriate way to cause light extraction from the light guide towards the side 510 opposite to the textured side, referred to as the output window.

One major advantage of the fourth and fifth preferred embodiments is that if the plastic material selected is flexible, the shape of the output window 408 or 510 can conform to non-flat lesions for an optimal treatment. Additionally, used in near-contact in the oral cavity, the device of the fourth or fifth preferred embodiment avoids having to shield the healthy tissue prior to treatment. Simulations show that the fourth and fifth preferred embodiments can achieve desired performance.

While five preferred embodiments and variations thereon have been disclosed above, those skilled in the art who have reviewed the present disclosure will readily appreciate that other embodiments can be realized within the scope of the invention. For example, disclosures of specific light sources are illustrative rather than limiting, as other light sources can be used, such as a fiber, an optics-terminated fiber such as a lens-terminated fiber or a fiber terminated with a diffractive optical element, an array of light-emitting diodes, or a combination of these. Also, one or more such light sources can be used. Moreover, some or all of the features of multiple embodiments can be combined. Therefore, the present invention should be construed as limited only by the appended claims.

We claim:

1. A device for irradiating an anatomic surface, the device comprising:
a light source; and
optics for causing light from the light source to be incident only on a portion of the anatomic surface, while shielding a remainder of the anatomic surface from the light.

2. The device of claim 1, wherein the optics comprise a body of oral impression material molded to conform to the anatomic surface and a reflective surface formed on the body of oral impression material, and wherein the light source is at least partially embedded in the body of oral impression material.

3. The device of claim 2, wherein the light source comprises one or more light sources selected from the group consisting of optical fibers, cylindrical diffusing tip optical fibers, optics-terminated optical fibers, and arrays of light emitting diodes.

4. The device of claim 1, wherein the optics comprise a light pipe having an input end, and wherein the light source is connected to the input end.

5. The device of claim 4, wherein the light pipe comprises at least one of a straight section, a tapered section, and a freeform section.

6. The device of claim 1, wherein the optics comprise:
an optical body having an output window and a hole for insertion of the source; and
a freeform reflector formed on a surface of the optical body.

7. The device of claim 6, wherein the light source comprises one or more light sources selected from the group consisting of optical fibers, cylindrical diffusing tip optical fibers, optics-terminated optical fibers, and arrays of light emitting diodes.

8. The device of claim 1, wherein the optics comprise a light guide having a first surface and a second surface, opposed to the first surface, having an output window, the first surface being textured to direct the light to the output window.

9. The device of claim 8, wherein the light guide is formed of a flexible material.

10. A method for irradiating an anatomic surface, the method comprising:
providing a device comprising a light source and optics for causing light from the light source to be incident only on a portion of the anatomic surface, while shielding a remainder of the anatomic surface from the light; and
using the device to cause the light to be incident only on the portion of the anatomic surface.

11. The method of claim 10, wherein the optics comprise a body of oral impression material molded to conform to the anatomic surface and a reflective surface formed on the body of oral impression material, and wherein the light source is at least partially embedded in a body of oral impression material.

12. The method of claim 11, wherein the light source comprises one or more light sources selected from the group consisting of optical fibers, cylindrical diffusing tip optical fibers, optics-terminated optical fibers, and arrays of light emitting diodes.

13. The method of claim 10, wherein the optics comprise a light pipe having an input end, and wherein the light source is connected to the input end.

14. The method of claim 13, wherein the light pipe comprises at least one of a straight section, a tapered section, and a freeform section.

15. The method of claim 10, wherein the optics comprise:
an optical body having an output window and a hole for insertion of the source; and
a freeform reflector formed on a surface of the optical body.

16. The method of claim 15, wherein the light source comprises one or more light sources selected from the group consisting of optical fibers, cylindrical diffusing tip optical fibers, optics-terminated optical fibers, and arrays of light emitting diodes.

17. The method of claim 10, wherein the optics comprise a light guide having a first surface and a second surface, opposed to the first surface, having an output window, the first surface being textured to direct the light to the output window.

18. The method of claim 17, wherein the light guide is formed of a flexible material.

19. A method for making a device for irradiating an anatomic surface, the method comprising:
(a) applying a body of oral impression material to the anatomic surface to conform the body of oral impression material to the anatomic surface;
(b) inserting a light source into the body of oral impression material; and
(c) forming a reflective surface on the body of oral impression material.

20. The method of claim 19, wherein the light source comprises one or more light sources selected from the group consisting of optical fibers, cylindrical diffusing tip optical fibers, optics-terminated optical fibers, and arrays of light emitting diodes.
